(19) United States
(12) Patent Application Publication
(10) Pub. No.: US 2023/0386614 A1
(43) Pub. Date: Nov. 30, 2023

(54) COMPOSITION SWEETNESS, BITTERNESS, UMAMI, LICORICE, LINGERING, SOURNESS OR SALTINESS PERCEPTION PREDICTION METHOD AND SYSTEM

(71) Applicant: Firmenich SA, Satigny (CH)

(72) Inventors: Eugénie CHANEL, Neuilly-Sur-Seine (FR); Bénédicte LE CALVE, Satigny (CH); Michelle SERGENT, Marseille (FR)

(21) Appl. No.: 18/322,140

(22) Filed: May 23, 2023

(30) Foreign Application Priority Data
May 24, 2022 (EP) 22174988.0
Aug. 9, 2022 (EP) 22189544.4

(51) Int. Cl. G16C 20/30

(52) U.S. Cl. CPC G16C 20/30

(57) ABSTRACT

The physical composition digital representation sweetness, bitterness, umami, licorice, lingering, sourness or saltiness perception prediction method (100) comprises:
a step (105) of inputting at least one physical flavoring ingredient digital representation identifier, said input defining a physical composition digital representation,
a step (110) of defining, for at least one physical flavoring ingredient digital representation identifier, a concentration of said physical flavoring ingredient in the physical composition digital representation,
a step (115) of calculating, as a function of at least one concentration defined, a value representative of the perceivable sweetness, bitterness, umami, licorice, lingering, sourness or saltiness of the input physical composition digital representation, said calculated value being representative of the physico-chemical activation of taste receptors by the physical composition corresponding to the composition digital representation, and
a step (120) of providing the calculated value.

### TECHNICAL FIELD OF THE INVENTION

The present invention relates to a physical composition digital representation sweetness, bitterness, umami, licorice, lingering, sourness or saltiness perception prediction method, a physical composition digital representation sweetness, bitterness, umami, licorice, lingering or saltiness perception prediction system, a physical composition digital representation sweetness, bitterness, umami, licorice, lingering or saltiness optimization method and a physical composition digital representation sweetness, bitterness, umami licorice, lingering or saltiness optimization system. It applies, in particular, to the fields of flavor design.

### BACKGROUND OF THE INVENTION

In current systems of flavor design, flavorists and technical experts run trial and error strategies to discover physical composition digital representations that exhibit a particular perceived sweetness, bitterness, umami, licorice, lingering, sourness or saltiness level or target. Such strategies waste considerable resources, including raw materials as well as human and computation time.

Indeed, sensory responses vary from individual to individual and thus, so far, no reliable and automatic predictors have been found. To capture the variability of sensory response, a significant number of measurements (sensory panels) are typically needed as the relationship from the taste perception of physical composition digital representations of physical flavoring ingredients as opposed to individual physical flavoring ingredients is not linear.

The fragrance and flavor (F&F) industry is constantly in search for new ingredients, novel perfumery and flavor applications, improved sensory experiences, and compounds that are more stable, biodegradable and non-toxic.

Because an aroma ingredient may interact with several olfactory receptors, it is often difficult to infer the sweetness, bitterness, umami, licorice, lingering, sourness or saltiness of physical flavoring compounds based on its chemical structure alone.

Typically, current models predict sensory descriptors such as “sweet”, “licorice” and “lingering” perceptions at any sucrose dosage in application. Such models allow to translate some application requirements (total Sweetness Brix Equivalency) into sensory attributes. Those models typically present a sigmoid shape. The function F(x) is the common structure of sensory attribute predictive functions. The input variable x refers to the log concentration of sucrose. Min and max are respectively minimum and maximum levels of observed sensory attribute; x50 is the log concentration at which the sensory attributes reaches half of its maximum value; β is representative of the slope of attribute increase.

$$F ( x ) = min + \frac{max - min}{1 + 1 {0}^{β ( x - {x}_{50} )}}$$

When multiple sweeteners/ingredients are present in an application, those models are not adequate anymore. Distinct behaviors of sweeteners, complexity of matrix as well as interactions between the very same sweeteners make such models unable to accurately predict the perceivable sweetness of a physical composition digital representation.

There remains a need to predict the at least one sweetness, bitterness, umami, licorice, lingering, sourness or saltiness perceivability of a given compound or physical composition digital representation and to facilitate new flavor ingredient discovery relevant to the F&F industry.

### SUMMARY OF THE INVENTION

The present invention is intended to remedy all or part of these disadvantages.

To this effect, according to a first aspect, the present invention aims at a physical composition digital representation sweetness, bitterness, umami, licorice, lingering, sourness or saltiness perception prediction method, comprising:
a step of inputting at least one physical flavoring ingredient digital representation identifier, upon a computer interface, said physical flavoring ingredient digital representation identifier being representative of a physical flavoring ingredient, said input defining a physical composition digital representation,
a step of defining, upon a computer interface, for at least one physical flavoring ingredient digital representation identifier, a concentration of said physical flavoring ingredient in the physical composition digital representation,
a step of calculating, by a computing system, as a function of at least one concentration defined, a value representative of the perceivable sweetness, bitterness, umami, licorice, lingering, sourness or saltiness of the input physical composition digital representation, said calculated value being representative of the physico-chemical activation of taste receptors by the physical composition corresponding to the composition digital representation, and
a step of providing, upon a computer interface, the calculated value representative of the perceivable sweetness, bitterness, umami, licorice, lingering, sourness or saltiness.

Such provisions allow for the accurate prediction of the perceivable sweetness, bitterness, umami, licorice, lingering, sourness or saltiness for a physical composition digital representation to be assembled. This allows considerable resource savings by allowing for dynamic flavor design.

In particular embodiments, the step of calculating is configured to operate a nth order polynomial function as a function of at least one defined concentration of physical flavoring ingredient in the physical composition digital representation.

Such provisions allow for the accurate perception prediction of the perceivable sweetness, bitterness, umami, licorice, lingering, sourness or saltiness for a physical composition digital representation to be assembled.

In particular embodiments, the method object of the present invention comprises:
a step of initialization, upon a computer interface, of physical flavoring ingredient digital representation identifiers, said input defining a physical composition digital representation,
a step of generating, by a computing system, a list of physical composition digital representation digital representation identifiers, each physical composition digital representation digital representation identifiers being representative of a physical composition digital representation,
a step of producing at least one generated physical composition digital representation,
a step of acquisition, upon a computer interface, of values representative of the perceived sweetness, bitterness, umami or saltiness of at least one generated physical composition digital representation and
a step of computing, by a computing system, coefficients in a nth order polynomial function using the input physical flavoring ingredient digital representation identifiers as variables,
   said coefficients being used during the step of calculating.

Such provisions allow for considerable resource savings by allowing for limited sample acquisition to create an accurate sweetness, bitterness, umami, licorice, lingering, sourness or saltiness perception prediction model.

In particular embodiments, the method object of the present invention comprises a step of setting, upon a computer interface, for at least one physical flavoring ingredient digital representation identifier, a value representative of the concentration of said physical flavoring ingredient in the input physical composition digital representation, the steps of producing and computing being performed as a function of said concentration.

Such provisions allow for the accurate perception prediction of the perceivable sweetness, bitterness, umami, licorice, lingering, sourness or saltiness for a physical composition digital representation to be assembled.

In particular embodiments, the method object of the present invention comprises, downstream of the step of generating, a step of physical composition digital representation digital representation identifier generation optimization, the step of producing being performed as a function of the output of said step of optimization.

Such provisions allow for the efficient selection of physical composition digital representations to be produced, allowing for the use of an optimized sample to generate the model.

In particular embodiments, the method object of the present invention comprises a step of constructing a database of physical flavoring ingredient interaction impact value upon sweetness, bitterness, umami, licorice, lingering, sourness or saltiness perception, said impact value:
being obtained as a function of the coefficients obtained during the step of computing and of the physical flavoring ingredient digital representation identifiers associated with these coefficients, and
used during the step of calculating.

Such embodiments allow for the constitution of a database of data representative of physical interactions between ingredients, said interactions being digitally represented by numerical values and used during later predictions.

In particular embodiments, the method object of the present invention comprises a step of determining, by a computing system, at least one replacement physical flavoring ingredient digital representation identifier for at least one input physical flavoring ingredient digital representation identifier, the step of providing being configured to provide the determined physical flavoring ingredient digital representation identifier.

Such provisions allow for the dynamic design of flavors, optimizing the perceivable sweetness, bitterness, umami, licorice, lingering, sourness or saltiness according to a determined target.

In particular embodiments, the physical composition digital representation saltiness, umami or bitterness perception prediction method object of the present invention comprises, downstream of the step of defining, a step of attributing, for at least one input physical flavoring ingredient digital representation identifier, a value for at least one attribute among:
a sodium chloride quantity,
a potassium chloride quantity,
a mono sodium glutamate quantity and/or
a ribotide quantity,
   at least one said value being used during the step of calculating to calculate a value representative of the perceivable saltiness, umami or bitterness of the input physical composition digital representation.

Such embodiments allow for the mapping of all physical flavoring ingredients into a four dimensional array, the dimensions of said array being directly linked to the saltiness, umami or bitterness perception of a fragrant ingredient and physical composition digital representation thereof.

In particular embodiments, the method object of the present invention comprises a step of selecting a physical composition application identifier for the physical composition, the step of calculating being configured to determine a prediction as a function of the physical composition application identifier selected.

Such embodiments allow for the accurate prediction of the behavior of a composition in a particular application (liquid, semi-liquid, solid, for example).

In particular embodiments, the method object of the present invention comprises a step of physical ingredients interaction parameter values database construction, in which at least two physical ingredients digital identifiers are associated to at least one physical interaction parameter value representative of a quantified synergistic, neutral or antagonistic interaction between the corresponding physical ingredients.

Such a parameter value may correspond to the nature of the interaction (synergistic, neutral or antagonistic) and/or to a value representative of the intensity of the nature of the interaction.

According to a second aspect, the present invention aims at a physical composition digital representation sweetness, bitterness, umami, licorice, lingering, sourness or saltiness optimization method, comprising:
a step of setting, upon a computer interface, a value representative of a desired perceivable sweetness, bitterness, umami, licorice, lingering, sourness or saltiness, minimum perceivable sweetness, bitterness, umami, licorice, lingering, sourness or saltiness or maximum perceivable sweetness, bitterness, umami, licorice, lingering, sourness or saltiness of a physical composition digital representation to be produced,
a step of determining, by a computing system, at least one physical flavoring ingredient digital representation identifier, upon a computer interface, said physical flavoring ingredient digital representation identifier being representative of a physical flavoring ingredient , said input defining a physical composition digital representation, said physical flavoring ingredient digital representation identifier being selected as a function of a value representative of the perceivable sweetness, bitterness, umami, licorice, lingering, sourness or saltiness associated to at least one physical flavoring ingredient digital representation identifier and the perceivable sweetness, bitterness, umami, licorice, lingering, sourness or saltiness set and
a step of providing, upon a computer interface, the determined physical flavoring ingredient digital representation identifiers.

Such provisions allow for the dynamic design of flavors, optimizing the selection of physical flavoring ingredients to be assembled to form a physical composition digital representation matching a determined target.

In particular embodiments, the method object of the present invention comprises:
a step of associating, by a computing system, at least one secondary attribute with at least one physical flavoring ingredient digital representation identifier and
a step of defining, for a physical composition digital representation to be produced, at least one threshold value for at least one secondary attribute, the step of determining being performed as a function of the secondary attribute associated with at least one physical flavoring ingredient digital representation identifier.

Such provisions allow for the dynamic design of flavors, optimizing the selection of physical flavoring ingredients to be assembled to form a physical composition digital representation matching a determined target as well as other secondary criteria.

In particular embodiments, at least one value set, during the step of setting corresponds to:
a total sweetness brix equivalent, referring to equivalent sucrose content in 100 g of product providing same sweetness perception than the one the physical composition digital representation to be determined,
a value representative of a maximum sucrose content
a maximum steviol equivalence.

Such provisions allow for optimal physical composition digital representation design matching thresholds fitting the abovementioned requirements.

In particular embodiments, the method object of the present invention comprises, downstream of the step of setting, a step of calculating a maximum value for at least one attribute among:
a sodium chloride quantity,
a potassium chloride quantity,
a mono sodium glutamate quantity and/or
a ribotide quantity,
   at least one said calculated value being used during the step of determining a physical composition digital representation.

Such embodiments allow for the determination of ingredients to form a physical composition digital representation based upon intermediate criteria associated to performance in saltiness, umami or bitterness perception.

In particular embodiments, any one of the methods object of the present invention comprises a step of assembling the physical composition represented by the physical composition digital representation .

Such provisions allow for the assembling of optimized physical compositions.

According to a third aspect, the present invention aims at a physical composition digital representation sweetness, bitterness, umami, licorice, lingering, sourness or saltiness perception prediction system, comprising:
means of inputting at least one physical flavoring ingredient digital representation identifier, said physical flavoring ingredient digital representation identifier being representative of a physical flavoring ingredient, said input defining a physical composition digital representation,
means of defining for at least one physical flavoring ingredient digital representation identifier, a concentration of said physical flavoring ingredient in the physical composition digital representation,
means of calculating, as a function of at least one concentration defined, a value representative of the perceivable sweetness, bitterness, umami or saltiness of the input physical composition digital representation, said calculated value being representative of the physico-chemical activation of taste receptors by the physical composition corresponding to the composition digital representation, and
means of providing the calculated value representative of the perceivable sweetness, bitterness, umami, licorice, lingering, sourness or saltiness.

The system object of the present invention provides for the same advantages as the corresponding method object of the present invention.

According to a fourth aspect, the present invention aims at a physical composition digital representation sweetness, bitterness, umami, licorice, lingering, sourness or saltiness optimization system, comprising:
means of setting a value representative of a desired perceivable sweetness, bitterness, umami, licorice, lingering, sourness or saltiness, minimum perceivable sweetness, bitterness, umami, licorice, lingering, sourness or saltiness or maximum perceivable sweetness, bitterness, umami, licorice, lingering, sourness or saltiness of a physical composition digital representation to be produced,
means of determining at least one physical flavoring ingredient digital representation identifier, upon a computer interface, said physical flavoring ingredient digital representation identifier being representative of a physical flavoring ingredient, said input defining a physical composition digital representation, said physical flavoring ingredient digital representation identifier being selected as a function of a value representative of the perceivable sweetness, bitterness, umami, licorice, lingering, sourness or saltiness associated to at least one physical flavoring ingredient digital representation identifier and the perceivable sweetness, bitterness, umami, licorice, lingering, sourness or saltiness set and
means of providing the determined physical flavoring ingredient digital representation identifiers.

The system object of the present invention provides for the same advantages as the corresponding method object of the present invention.

### BRIEF DESCRIPTION OF THE DRAWINGS

Other advantages, purposes and particular characteristics of the invention shall be apparent from the following non-exhaustive description of at least one particular method or system which is the object of this invention, in relation to the drawings annexed hereto, in which:

FIG. 1 represents, schematically, a first particular succession of steps of the method subject of the present invention,

FIG. 2 represents, schematically, a second particular succession of steps of the method subject of the present invention,

FIG. 3 represents, schematically, a third particular succession of steps of the method subject of the present invention,

FIG. 4 represents, schematically, a fourth particular succession of steps of the method subject of the present invention and

FIG. 5 represents, schematically, a first particular embodiment of the system subject of the present invention,

FIG. 6 represents, schematically, a second particular embodiment of the system subject of the present invention, and

FIG. 7 represents, schematically, a particular embodiment of a computer system susceptible of implementing embodiments of the present invention.

### DETAILED DESCRIPTION OF THE INVENTION

This description is not exhaustive, as each feature of one embodiment may be combined with any other feature of any other embodiment in an advantageous manner.

Various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood as inclusive.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

It should be noted at this point that the figures are not to scale.

As used herein, the terms “volatile ingredient” designate any ingredient, preferably presenting a physical flavoring or fragrance capacity. The terms “compound” or “ingredient” designate the same items as “volatile ingredient.” An ingredient may be formed of one or more chemical molecules.

The terms “formula” or physical composition digital representation designates a liquid, solid or gaseous assembly of at least one volatile ingredient.

As used herein, a “flavor” refers to the olfactory perception resulting from the sum of odorant receptor(s) activation, enhancement, and inhibition (when present) by at least one volatile ingredient via orthonasal and retronasal olfaction as well as activation of the taste buds which contain taste receptor cells. Accordingly, by way of illustration and by no means intending to limit the scope of the present disclosure, a “flavor” results from the olfactory and taste bud perception arising from the sum of a first volatile ingredient that activates an odorant receptor or taste bud associated with a coconut tonality, a second volatile ingredient that activates an odorant receptor or taste bud associated with a celery tonality, and a third volatile ingredient that inhibits an odorant receptor or taste bud associated with a hay tonality.

In the present invention, a “sweetener” (a type of physical flavoring ingredient) is a common saccharide sweeteners, such as sucrose, fructose, glucose, and sweetener physical composition digital representations comprising natural sugars, such as corn syrup (including high fructose corn syrup) or other syrups or sweetener concentrates derived from natural fruit and vegetable sources. In some embodiments, the sweetener is sucrose, fructose, or a combination thereof. In some embodiments, the sweetener is sucrose. In some other embodiments, the sweetener is selected from rare natural sugars including D-allose, D-psicose, L-ribose, D-tagatose, L-glucose, L-fucose, L-arbinose, D-turanose, and D-leucrose.

In some embodiments, a sweetener is selected from semi-synthetic “sugar alcohol” sweeteners such as erythritol, isomalt, lactitol, mannitol, sorbitol, xylitol, maltodextrin, and the like. In some embodiments, a sweetener is selected from artificial sweeteners such as aspartame, saccharin, acesulfame-K, cyclamate, sucralose, and alitame. In some embodiments, a sweetener is selected from the group consisting of cyclamic acid, mogroside, tagatose, maltose, galactose, mannose, sucrose, fructose, lactose, allulose neotame and other aspartame derivatives, glucose, D-tryptophan, glycine, maltitol, lactitol, isomalt, hydrogenated glucose syrup (HGS), hydrogenated starch hydrolyzate (HSH), stevioside, rebaudioside A, other sweet Stevia-based glycosides, chemically modified steviol glycosides (such as glucosylated steviol glycosides), mogrosides, chemically modified mogrosides (such as glucosylated mogrosides), carrelame and other guanidine-based sweeteners. In some embodiments, a sweetener is a combination of two or more of the sweeteners set forth in this paragraph. In some embodiments, a sweetener may combinations of two, three, four or five sweeteners as disclosed herein. In some embodiments, a sweetener may be a sugar. In some embodiments, the sweetener may be a combination of one or more sugars and other natural and artificial sweeteners. In some embodiments, a sweetener is a sugar. In some embodiments, the sugar is cane sugar. In some embodiments, the sugar is beet sugar. In some embodiments, the sugar may be sucrose, fructose, glucose or combinations thereof. In some embodiments, the sugar may be sucrose. In some embodiments, the sugar may be a combination of fructose and glucose.

The sweetener can also include, for example, sweetener physical composition digital representations comprising one or more natural or synthetic carbohydrate, such as corn syrup, high fructose corn syrup, high maltose corn syrup, glucose syrup, sucralose syrup, hydrogenated glucose syrup (HGS), hydrogenated starch hydrolyzate (HSH), or other syrups or sweetener concentrates derived from natural fruit and vegetable sources, or semi synthetic “sugar alcohol” sweeteners such as polyols. Non-limiting examples of polyols in some embodiments include erythritol, maltitol, mannitol, sorbitol, lactitol, xylitol, isomalt, propylene glycol, glycerol (glycerin), threitol, galactitol, palatinose, reduced isomalto-oligosaccharides, reduced xylo-oligosaccharides, reduced gentio-oligosaccharides, reduced maltose syrup, reduced glucose syrup, isomaltulose, maltodextrin, and the like, and sugar alcohols or any other carbohydrates or combinations thereof capable of being reduced which do not adversely affect taste.

A sweetener may be a natural or synthetic sweetener that includes, but is not limited to, agave inulin, agave nectar, agave syrup, amazake, brazzein, brown rice syrup, coconut crystals, coconut sugars, coconut syrup, date sugar, fructans (also referred to as inulin fiber, fructo-oligosaccharides, or oligo-fructose), green stevia powder, stevia rebaudiana, rebaudioside A, rebaudioside B, rebaudioside C, rebaudioside D, rebaudioside E, rebaudioside F, rebaudioside I, rebaudioside H, rebaudioside L, rebaudioside K, rebaudioside J, rebaudioside N, rebaudioside O, rebaudioside M and other sweet stevia-based glycosides, stevioside, stevioside extracts, honey, Jerusalem artichoke syrup, licorice root, luo han guo (fruit, powder, juice concentrates, or extracts), lucuma (fruit, powder, or extracts), maple sap (including, for example, sap extracted from Acer saccharum, Acer nigrum, Acer rubrum, Acer saccharinum, Acer platanoides, Acer negundo, Acer macrophyllum, Acer grandidentatum, Acer glabrum, Acer mono), maple syrup, maple sugar, walnut sap (including, for example, sap extracted from Juglans cinerea, Juglans nigra, Juglans ailatifolia, Juglans regia), birch sap (including, for example, sap extracted from Betula papyrifera, Betula alleghaniensis, Betula lenta, Betula nigra, Betula populifolia, Betula pendula), sycamore sap (such as, for example, sap extracted from Platanus occidentalis), ironwood sap (such as, for example, sap extracted from Ostrya virginiana), mascobado, molasses (such as, for example, blackstrap molasses), molasses sugar, monatin, monellin, cane sugar (also referred to as natural sugar, unrefined cane sugar, or sucrose), palm sugar, panocha, piloncillo, rapadura, raw sugar, rice syrup, sorghum, sorghum syrup, cassava syrup (also referred to as tapioca syrup), thaumatin, yacon root, malt syrup, barley malt syrup, barley malt powder, beet sugar, cane sugar, crystalline juice crystals, caramel, carbitol, carob syrup, castor sugar, hydrogenated starch hydrolates, hydrolyzed can juice, hydrolyzed starch, invert sugar, anethole, arabinogalactan, arrope, syrup, P-4000, acesulfame potassium (also referred to as acesulfame K or ace-K), alitame (also referred to as aclame), advantame, aspartame, baiyunoside, neotame, benzamide derivatives, bernadame, canderel, carrelame and other guanidine-based sweeteners, vegetable fiber, corn sugar, coupling sugars, curculin, cyclamates, cyclocarioside I, demerara, dextran, dextrin, diastatic malt, dulcin, sucrol, valzin, dulcoside A, dulcoside B, emulin, enoxolone, maltodextrin, saccharin, estragole, ethyl maltol, glucin, gluconic acid, glucono-lactone, glucosamine, glucoronic acid, glycerol, glycine, glycyphillin, glycyrrhizin, glycyrrhetic acid monoglucuronide, golden sugar, yellow sugar, golden syrup, granulated sugar, gynostemma, hernandulcin, isomerized liquid sugars, jallab, chicory root dietary fiber, kynurenine derivatives (including N′-formyl-kynurenine, N′-acetyl-kynurenine, 6-chloro-kynurenine), galactitol, litesse, ligicane, lycasin, lugduname, guanidine, falernum, mabinlin I, mabinlin II, maltol, maltisorb, maltodextrin, maltotriol, mannosamine, miraculin, mizuame, mogrosides (including, for example, mogroside IV, mogroside V, and neomogroside), mukurozioside, nano sugar, naringin dihydrochalcone, neohesperidine dihydrochalcone, nib sugar, nigero-oligosaccharide, norbu, orgeat syrup, osladin, pekmez, pentadin, periandrin I, perillaldehyde, perillartine, petphyllum, phenylalanine, phlomisoside I, phlorodizin, phyllodulcin, polyglycitol syrups, polypodoside A, pterocaryoside A, pterocaryoside B, rebiana, refiners syrup, rub syrup, rubusoside, selligueain A, shugr, siamenoside I, siraitia grosvenorii, soybean oligosaccharide, Splenda, SRI oxime V, steviol glycoside, steviolbioside, stevioside, strogins 1, 2, and 4, sucronic acid, sucrononate, sugar, suosan, phloridzin, superaspartame, tetrasaccharide, threitol, treacle, trilobtain, tryptophan and derivatives (6-trifluoromethyl-tryptophan, 6-chloro-D-tryptophan), vanilla sugar, volemitol, birch syrup, aspartame-acesulfame, assugrin, and combinations or blends of any two or more thereof. Such naturally occurring sweeteners, such as naturally occurring rebaudiosides, may be obtained in any suitable way, for example, by extracting the compounds from plants, by enzymatic bioconversion, or by fermentation.

In still other embodiments, a sweetener can be a chemically or enzymatically modified natural high potency sweetener. Modified natural high potency sweeteners include glycosylated natural high potency sweetener such as glucosyl-, galactosyl-, or fructosyl-derivatives containing 1-50 glycosidic residues. Glycosylated natural high potency sweeteners may be prepared by enzymatic transglycosylation reaction catalyzed by various enzymes possessing transglycosylating activity. In some embodiments, a modified sweetener can be substituted or unsubstituted.

Additional sweeteners also include combinations of any two or more of any of the aforementioned sweeteners. In some embodiments, a sweetener may comprise combinations of two, three, four or five sweeteners as disclosed herein. In some embodiments, a sweetener may be a sugar. In some embodiments, the sweetener may be a combination of one or more sugars and other natural and artificial sweeteners. In some embodiments, a sweetener is a caloric sweetener, such as sucrose, fructose, xylitol, erythritol, or combinations thereof. In some embodiments, the ingestible physical composition digital representations are free (or, in some embodiments) substantially free of stevia-derived sweeteners, such as steviol glycosides, glucosylated steviol glycosides, or rebaudiosides.

As used herein, the term “taste receptor” refers to receptors embedded in the plasma membrane of taste cells that bind taste molecules including sweet, bitter, salty, sour and umami compounds as well as fatty acids. The Taste receptor may be one or more members of a family of G protein-coupled receptors (GPCRs) with seven-transmembrane domains that are expressed in taste cells. The binding of taste molecules leads to the activation of taste receptors, which triggers signals and signal transduction. Perception of basic taste qualities including sweet, bitter, salty, fatty, sour, umami as well as of somatosensory sensory qualities including pungency, temperature, touch, pressure, texture and other tactile stimuli are sensed and brought about by taste GPCRs (sweet, bitter, umami/amino acids, fatty acids) as well as ion channels (e.g. salty and sour taste, pungency, temperature) and molecules involved in transport of taste molecules such as e.g. fatty acid scavengers including CD36. G-protein coupled receptors (GPCRs) represent the largest family of cell surface receptors with an estimated number of up to 1000 genes within the human genome characterized by a seven-transmembrane configuration as their main feature. (Bockaert and Pin, 1999; Pierce et al., 2002). GPCRs are activated by a multitude of different ligands, including peptides, proteins, lipids, small molecules, ions or even photons. Activated GPCRs alter their conformation allowing it to catalyze the exchange of guanosine diphosphate (GDP) for guanosine triphosphate (GTP) on the -subunit of a heterotrimeric g-protein coupled to the GPCR.

As used herein, the terms “means of inputting” is, for example, a keyboard, mouse and/or touchscreen adapted to interact with a computing system in such a way to collect user input. In variants, the means of inputting are logical in nature, such as a network port of a computing system configured to receive an input command transmitted electronically. Such an input means may be associated to a GUI (Graphic User Interface) shown to a user or an API (Application programming interface). In other variants, the means of inputting may be a sensor configured to measure a specified physical parameter relevant for the intended use case.

As used herein, the terms “computing system” or “computer system” designate any electronic calculation device, whether unitary or distributed, capable of receiving numerical inputs and providing numerical outputs by and to any sort of interface, digital and/or analog. Typically, a computing system designates either a computer executing a software having access to data storage or a client-server architecture wherein the data and/or calculation is performed at the server side while the client side acts as an interface.

As used herein, the terms “digital representation identifier” refer to any computerized representation identifier, such as one used in a computer database, representing a physical object, such as a physical flavoring ingredient. A digital representation identifier may refer to a label representative of the name, chemical structure or internal reference of the physical flavoring ingredient. Such a representation is bijective, meaning that one physical flavoring ingredient corresponds to one physical flavoring ingredient digital representation identifier and vice versa.

As used herein, the term “perception prediction” refers to the capacity to calculate a sensory quantification which is the translation of a biological/physiological reaction of sensory receptors of a user to a composition of physical flavoring ingredients.

In the present description, the terms ‘materialized’ or ‘physical’ is intended as existing outside of the digital environment of the present invention. ‘Materialized’ or ‘physical’ may mean, for example, readily found in nature or synthesized in a laboratory or chemical plant. In any event, a materialized physical composition digital representation presents a tangible reality. The terms ‘to be compounded’ or ‘compounding’ refer to the act of materialization of a physical composition digital representation, whether via extraction and assembly of ingredients or via synthetization and assembly of ingredients.

For the sake of clarity, by “flavored consumer product” or “application”, it is meant to designate an edible product or oral composition such as, for example, pharmaceutical compositions, edible gel mixes and compositions, dental compositions, foodstuffs beverages and beverage products. The flavored consumer product may be in a different form. A non-exhaustive list of suitable form of the consumer product may include fried, frozen, marinated, battered, chilled, dehydrated, powder blended, canned, reconstituted, retorted, baked, cooked, fermented, microfiltred, pasteurized, blended or preserved. Therefore, a flavored consumer product according to the invention comprises the invention's composition, as well as optional benefit agents, corresponding to taste and flavor profile of the desired edible product, e.g. a cream dessert.

The nature and type of the constituents of the foodstuffs or beverages do not warrant a more detailed description here, the skilled person being able to select them on the basis of his general knowledge and according to the nature of said product.

Typical examples of said flavored consumer product include:
baked goods (e.g. breads, dry biscuits, cakes, rice cakes, rice crackers, cookies, crackers, donuts, muffins, pastries, pre-mixes, other baked goods),
non-alcoholic beverages (e.g. aqueous beverages, enhanced/slightly sweetened water drinks, flavored carbonated and still mineral and table waters, carbonated soft drinks, non-carbonated beverages, carbonated waters, still waters, softs, bottled waters, sports/energy drinks, juice drinks, vegetable juices, vegetable juice preparations, broth drinks),
alcoholic beverages (e.g. beer and malt beverages, spirituous beverages, wines, liquors),
instant or ready-to-drink beverages (e.g. instant vegetable drinks, powdered soft drinks, instant coffees and teas, black teas, green teas, oolong teas, herbal infusions, cacaos (e.g. water- based), tea-based drinks, coffee-based drinks, cacao-based drinks, infusions, syrups, frozen fruits, frozen fruit juices, water-based ices, fruit ices, sorbets),
confectionary products (e.g. filings, toppings, chewing gums, hard and soft candies),
chocolate and coating products (e.g. chocolates, spreads and coverture product containing sugar and/or cocoa butter and/or vegetable oil(s)),
products based on fat and oil or emulsions thereof (e.g. mayonnaises, spreads, regular or low fat margarines, butter/margarine blends, flavored oils, shortenings, remoulades, dressings, salad dressings, spice preparations, peanut butters),
desserts (e.g. gelatins, puddings, dessert creams),
vegetable preparations (e.g. ketchups, sauces, processed and reconstituted vegetables, dried vegetables, deep frozen vegetables, pre-cooked vegetables, vegetables pickled in vinegar, vegetable concentrates or pastes, cooked vegetables, potato preparations, vegetable juices), and/or
ready dishes (e.g. instant noodles, rice, pastas, pizzas, tortillas, wraps) and soups and broths (e.g. stock, savory cubes, dried soups, instant soups, pre-cooked soups, retorted soups), sauces (instant sauces, dried sauces, ready-made sauces, gravies, sweet sauces, a relish sauces, a sour sauces).

It should be noted that an achievement of the present invention is the capacity to predict the perceived psychophysical intensity regarding particular olfactory or taste descriptors (sweetness, bitterness, umami, licorice, lingering, sourness or saltiness) for physical compositions. Such a capacity is obtained thanks to the accurate digital representation of the impact of individual physical flavoring ingredients. Such a digital representation may be directly predicted based upon the addition of the individual physical flavoring ingredients to the physical composition digital representation or based upon the transposition of said physical flavoring ingredients in a dimensional space where the dimensions correspond to equivalent ingredients, the impact of said equivalent ingredient upon the olfactory or taste descriptors being known. The latter proposition does not require the physical flavoring ingredient to be formed of said equivalent ingredients but only that a certain ratio of perceivability may be established between the physical flavoring ingredient and the equivalent ingredients.

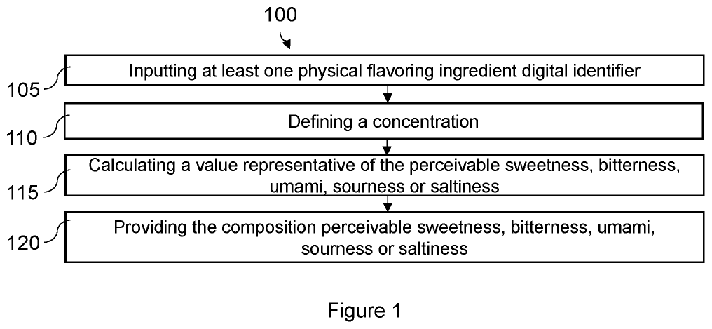

FIG. 1 shows a particular succession of steps of the method 100 object of the present invention. This physical composition digital representation sweetness, bitterness, umami, licorice, lingering, sourness or saltiness perception prediction method 100, comprises:
a step 105 of inputting at least one physical flavoring ingredient digital representation identifier, upon a computer interface, said physical flavoring ingredient digital representation identifier being representative of a physical flavoring ingredient, said input defining a physical composition digital representation,
a step 110 of defining, upon a computer interface, for at least one physical flavoring ingredient digital representation identifier, a concentration of said physical flavoring ingredient in the physical composition digital representation,
a step 115 of calculating, by a computing system, as a function of at least one concentration defined, a value representative of the perceivable sweetness, bitterness, umami or saltiness of the input physical composition digital representation, said calculated value being representative of the physico-chemical activation of taste receptors by the physical composition corresponding to the composition digital representation and
a step 120 of providing, upon a computer interface, the calculated value representative of the perceivable sweetness, bitterness, umami or saltiness.

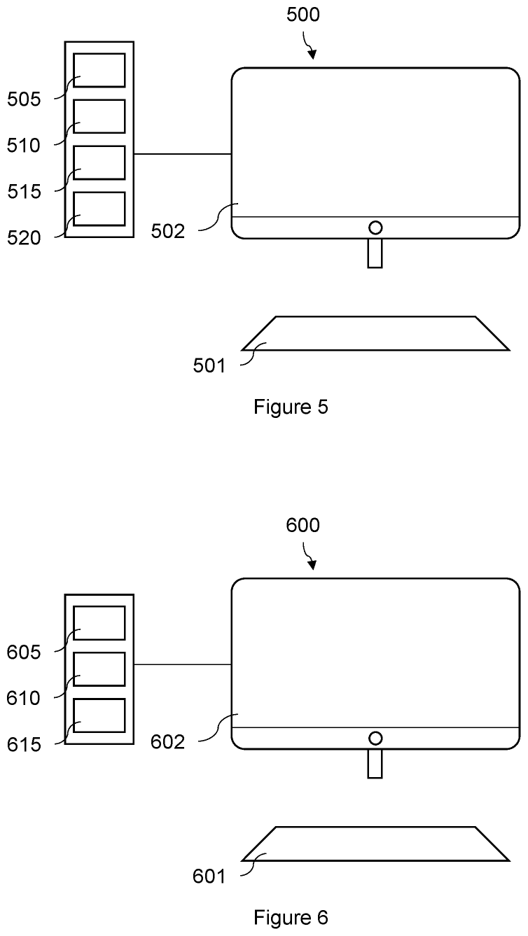

The step 105 of inputting is performed, for example, by means 505 of inputting such as shown in FIG. 5. Such means 505 of inputting are, for example, a computer software executed upon a computing device, said software presenting controller characteristics for a computer interface, such as a keyboard 501 for example. Any type of computer interface may be used for this step 105 of inputting, including but not limited to API inputs.

In particular embodiments, the step 105 of inputting is performed via a graphic user interface (“GUI”), displaying on a screen 502 a selection of physical flavoring ingredient digital representation identifiers to be selected by a user to create a physical composition digital representation. Such physical flavoring ingredient digital representation identifiers may also be searched via a search engine upon the GUI.

The step 115 of calculating is performed, for example, by means 510 of calculating such as shown in FIG. 6. Such means 510 of calculating are, for example, a computer software executed by a computing device, such software being configured to execute instructions representative of the step 115 of calculating. Many mathematical formulas may be used to implement the step 115 of calculating, with varying degree of performance.
In particular embodiments, the step 115 of calculating is configured to operate a nth order polynomial function as a function of at least one defined concentration of physical flavoring ingredient in the physical composition digital representation. A nth order polynomial function is a polynomial function with one or more variables in which the highest-degree term is of the second degree, each variable being associated with a coefficient.

For example, such a polynomial function may be, for a two physical flavoring ingredients physical composition digital representation in the context of a sweetness perceivability application:

Sweetness=b₀+b₁Ing1+b₂Ing2+b₁₁Ing1²+b₂₂Ing2²+b₁₂Ing1*Ing2

Where:
Ing1 and Ing2 are normalized concentrations of the ingredients
bₓ are the model parameters, representing the effect of input physical flavoring ingredients as well as their potential interactions (which can be synergetic, neutral or antagonist).

Such model parameters, or coefficients, may be application dependent, such as Flavored Water, Iced Tea, Carbonated Soft Drinks, Flavored Milk and Drinkable Yoghurt.

Such model parameters may be constrained, for example, in the context of flavored water for North American tastes:
Brix equivalent of the physical composition digital representation,
Perceived sweetness of the physical composition digital representation,
Sugar content of the physical composition digital representation,
Steviol equivalence of the physical composition digital representation,
Lingering perception of the physical composition digital representation (to be minimized) and
Licorice perception of the physical composition digital representation (to be minimized).

In particular embodiments, the method 200 object of the present invention comprises a step of selecting a value representative of a target application for the physical composition digital representation, the step 115 of calculating being performed as a function of the selected target application.

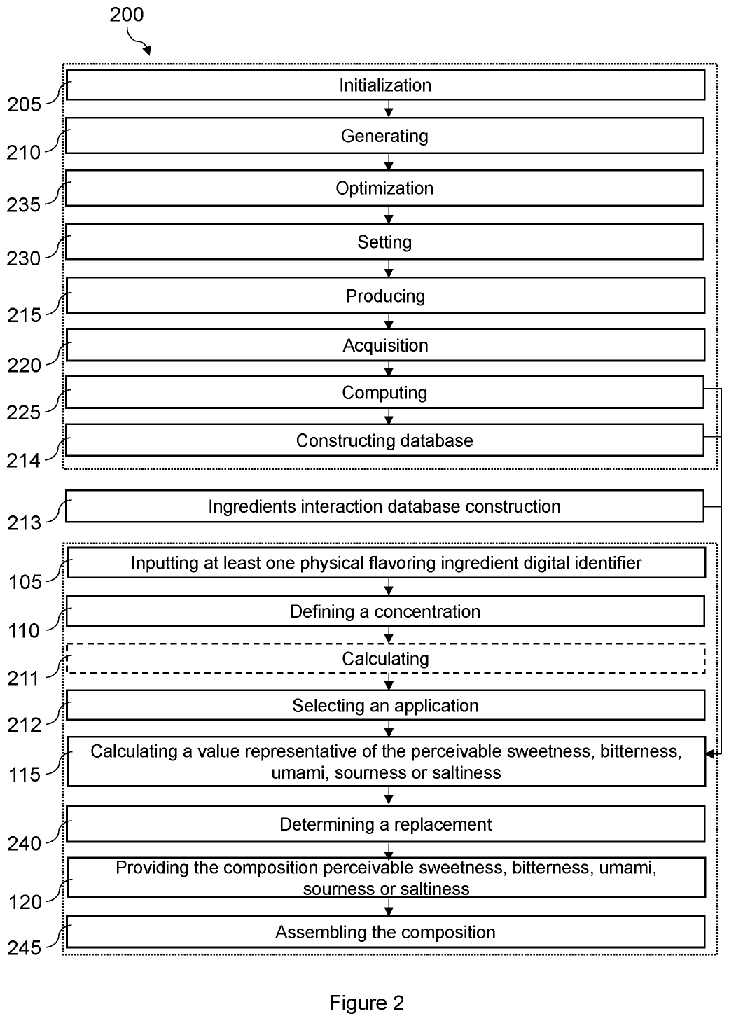

Such model parameters, or coefficients, may be obtained prior to the execution of the steps of inputting 105, defining 110 and calculating 115, by executing the following steps which aim at defining the model parameters to be used in the perception prediction or optimization algorithms object of the present invention. Such steps are shown in FIG. 2 as such:
a step 205 of initialization, upon a computer interface, of physical flavoring ingredient digital representation identifiers, said input defining a physical composition digital representation,
a step 210 of generating, by a computing system, a list of physical composition digital representation digital representation identifiers, each physical composition digital representation digital representation identifiers being representative of a physical composition digital representation,
a step 215 of producing at least one generated physical composition digital representation,
a step 220 of acquisition, upon a computer interface, of values representative of the perceived sweetness, bitterness, umami or saltiness of at least one generated physical composition digital representation and
a step 225 of computing, by a computing system, coefficients in a nth order polynomial function using the input physical flavoring ingredient digital representation identifiers as variables,
   said coefficients being used during the step 115 of calculating.

The model parameters and the value of such model parameters, or coefficients, may be application dependent. This means that if the method 200 comprises a step of selecting a value representative of a target application for the physical composition digital representation, the step 115 of calculating uses the parameters and values for such parameters that correspond to the selected target application.

The step 205 of initialization is performed, for example, by means of initialization, such as a computer program run by a computing system. During this step 205 of initialization, any input means may be used by a user to select physical flavoring ingredient digital representation identifiers.

The step 210 of generating is performed, for example, by a computer program run upon a computing a device. During this step 210 of generating, at least one physical composition digital representation digital representation identifier is generated. The objective of this step 210 of generating is to reduce the initial sample size to a smaller but representative sample size.

In simple embodiments, during the step 210 of generating, random combinations of physical flavoring ingredients are generated.

In preferred embodiments, during the step 210 of generating, combinations of physical flavoring ingredients are generated in an optimized manner so as to reduce de number of physical composition digital representations to produce in order to generate the model during the step 225 of computing.

In preferred embodiments, the method 200 object of the present invention comprises a step 214 of constructing a database of physical flavoring ingredient interaction impact value upon sweetness, bitterness, umami, licorice, lingering, sourness or saltiness perception, said impact value:
being obtained as a function of the coefficients obtained during the step 225 of computing and of the physical flavoring ingredient digital representation identifiers associated with these coefficients, and
used during the step 115 of calculating.

The step 214 of constructing a database may be performed, for example, manually, semi-automatically or automatically by interacting with a memory of any type and storing, within this memory, information representative of the physical flavoring ingredient digital representation identifiers associated with the computed coefficients and said coefficients or representations of said coefficients.

In particular embodiments, the method 200 object of the present invention comprises, downstream of the step 210 of generating, a step 235 of physical composition digital representation digital representation identifier generation optimization, the step 215 of producing being performed as a function of the output of said step of optimization.

In such embodiments, the step 235 of optimization may use a d-optimal algorithm.

D-optimal algorithms are straight optimizations based on a chosen optimality criterion and the model that will be fit. The optimality criterion used in generating D-optimal algorithms is one of maximizing the determinant of the information matrix.

This optimality criterion results in minimizing the generalized variance of the parameter estimates for a pre-specified model. As a result, the optimality of a given D-optimal algorithm is model dependent. That is, the experimenter must specify a model for the design before a computer can generate the specific treatment combinations.

Given the total number of treatment runs for an experiment and a specified model, the computer algorithm chooses the optimal set of design runs from a candidate set of possible design treatment runs. This candidate set of treatment runs usually consists of all possible combinations of various factor levels that one wishes to use in the experiment.

In other words, the candidate set is a collection of physical composition digital representations from which the D-optimal algorithm chooses the physical composition digital representations to include in the design. The computer algorithm generally uses a stepping and exchanging process to select the set of physical composition digital representations runs.

The step 215 of producing is performed, for example, by using any means to assemble a physical composition digital representation known to a person skilled in the art. Such means of assembly include, for example, laboratory materials or manufacturing plants.

The step 220 of acquisition is performed, for example, by a computer program run upon a computing a device. During this step 220 of acquisition, the psychophysical intensity of the sweetness, bitterness, umami or saltiness of the physical composition digital representation, perceived by at least one user, is recorded upon a computer interface linked to a database for example.

The step 225 of computing is performed, for example, by a computer program run upon a computing a device. During this step 225 of computing, the coefficients in a nth order polynomial function representative of the interactions between physical flavoring ingredients are obtained.

This is performed, for example, by a implementing a d-optimal algorithm which allows to select right physical composition digital representations on which it is needed to measure sweetness, licorice, lingering or other sensory perception.

Then, from these measurements, the coefficients of the model are identified by fitting a multiple linear model to the data.

In particular embodiments, such as shown in FIG. 2, the method 200 object of the present invention comprises a step 230 of setting, upon a computer interface, for at least one physical flavoring ingredient digital representation identifier, a value representative of the concentration of said physical flavoring ingredient in the input physical composition digital representation, the steps of producing 215 and computing 225 being performed as a function of said concentration.

The step 230 of setting is performed, for example, by a computer program run upon a computing device. During this step 230 of setting, a user is for example prompted with a query on a GUI to input concentration values for the selected physical flavoring ingredient digital representation identifiers.

The step 120 of providing is performed, for example, by means 520 of providing such as shown in FIG. 5. Such means 520 of providing is, for example, a computer software executed upon a computing device, said software presenting controller characteristics for a computer interface, such as a computer screen 502 for example. Any type of computer interface may be used for this step 120 of providing, including but not limited to API outputs.

In more advanced embodiments, such as shown in FIG. 2, the method 200 object of the present invention comprises a step 240 of determining, by a computing system, at least one replacement physical flavoring ingredient digital representation identifier for at least one input physical flavoring ingredient digital representation identifier, the step 120 of providing being configured to provide the determined physical flavoring ingredient digital representation identifier.

The step 240 of determining is performed, for example, by a computer program run upon a computing device. During such a step 240 of determining, candidate physical flavoring ingredients that can act as a replacement for at least one input physical flavoring ingredient are evaluated. Such an evaluation may take the form of the calculation, performed similarly to the step 115 of calculating, of the perceivable sweetness, bitterness, umami, licorice, lingering, sourness or saltiness should the candidate physical flavoring ingredient replace at least one input physical flavoring ingredient in the physical composition digital representation. The result of this calculation may then be compared to the previously calculated perceivable sweetness, bitterness, umami, licorice, lingering, sourness or saltiness or compared to a predetermined or user-set threshold and, depending on the result, the candidate physical flavoring ingredient digital representation identifier may be presented during the step 120 of providing.

In particular, certain attributes associated with candidate ingredients may be minimized (licorice or lingering perception for example) or maximized (umami or bitterness perception for example). For example, if two ingredients provide the same perceivable sweetness and one of them provides a lower licorice perception, then the ingredient with the lower licorice perception is selected.

In particular embodiments, such as shown in FIG. 2, the method 200 object of the present invention comprises a step 245 of assembling the physical composition represented by the physical composition digital representation. Such a step 245 of assembling may be performed according to any method known to a person skilled in the art of assembling physical flavoring chemical physical composition digital representations.

In particular embodiments, such as shown in FIG. 2, the method 200 object of the present invention comprises a step 212 of selecting a physical composition application identifier for the physical composition, the step 115 of calculating being configured to determine a prediction as a function of the physical composition application identifier selected.

Such a step 212 of selecting may be performed by using any means of inputting an application identifier value for a defined digital representation of a composition.

Such an application identifier may correspond to, for example:
liquid,
semi-liquid,
solid,
semi-solid,
soft drink, or
soup.

The selection of such an application identifier may result in the use of different parameter values during the step 115 of calculating. Such parameter values may correspond to, for example, different coefficients, for identical physical ingredients, in a nth order polynomial function or any other mathematical function representing the physical interactions between physical ingredients in the associated application. Such a parameter value may even correspond to a change in mathematical function depending on the application identifier, said mathematical function better representing the physical interactions between physical ingredients in the associated application.

Such parameter values may be obtained by physically measuring the interactions between pairs or larger groups of physical ingredients in the context of one said application and by constitution a database of physical ingredients interaction parameter values for each said application and for at least one of perceivable sweetness, bitterness, umami, licorice, lingering, sourness or saltiness, minimum perceivable sweetness, bitterness, umami, licorice, lingering, sourness or saltiness or maximum perceivable sweetness, bitterness, umami, licorice, lingering, sourness or saltiness.

The mathematical function representing the physical interactions between physical ingredients in an associated application can be determined similarly to the mathematical function exemplified in the present document.

In particular embodiments, such as shown in FIG. 2, the method 200 object of the present invention comprises a step 213 of physical ingredients interaction parameter values database construction, in which at least two physical ingredients digital identifiers are associated to at least one physical interaction parameter value representative of a quantified synergistic, neutral or antagonistic interaction between the corresponding physical ingredients.

Such a database can be used during the step 115 of calculating to determine the perceivable sweetness, bitterness, umami, licorice, lingering, sourness or saltiness.

In particular embodiments, such as shown in FIG. 2, the method 200 is adapted for physical composition digital representation saltiness, umami or bitterness perception prediction. In such embodiments, the method 200 comprises, downstream of the step 110 of defining, a step 211 of attributing, for at least one input physical flavoring ingredient digital representation identifier, a value for at least one attribute among:
a sodium chloride quantity,
a potassium chloride quantity,
a mono sodium glutamate quantity and/or
a ribotide quantity,
   at least one said value being used during the step 215 of calculating to calculate a value representative of the perceivable saltiness, umami or bitterness of the input physical composition digital representation.

The step 211 of attributing is performed, for example, by a computer program run upon a computing device. During such a step 211 of attributing, values for the attributes, or dimensions, or parameters, or coefficients, of the model are set on the basis of a known correspondence between physical flavoring ingredient and attribute values. Such correspondence may be retrieved, for example, from a database linking physical flavoring ingredient digital representation identifiers to attribute values. In such embodiments, the saltiness, umami or bitterness may be calculated using the following equation:

Saltiness=b₀+b₁NaCl+b₂KCl+b₃MSG=b₄Ribo+b₁₁NaCl²+b₂₂KCl²+b₃₃MSG²+b₄₄Ribo²+b₁₂NaCl*KCl+b₁₃NaCl*MSG+b₁₄NaCl*Ribo+b₂₃KCl*MSG+b₂₄KCl*Ribo+b₃₄MSG*Ribo

NaCl, KCl, MSG and Ribotide being the normalized concentration of sodium chloride, potassium chloride, monosodium glutamate and ribotide.

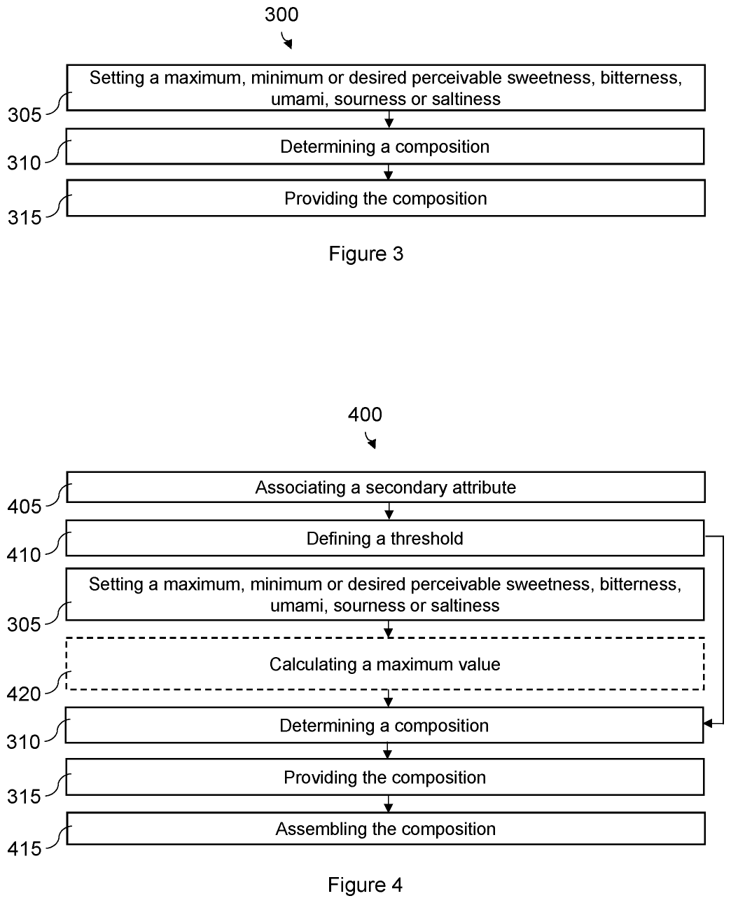

FIG. 3 shows a particular succession of steps of the method 300 object of the present invention. This physical composition digital representation sweetness, bitterness, umami or saltiness optimization method 300 comprises:
a step 305 of setting, upon a computer interface, a value representative of a desired perceivable sweetness, bitterness, umami, licorice, lingering, sourness or saltiness, minimum perceivable sweetness, bitterness, umami, licorice, lingering, sourness or saltiness or maximum perceivable sweetness, bitterness, umami, licorice, lingering, sourness or saltiness of a physical composition digital representation to be produced,
a step 310 of determining, by a computing system, at least one physical flavoring ingredient digital representation identifier, upon a computer interface, said physical flavoring ingredient digital representation identifier being representative of a physical flavoring ingredient, said input defining a physical composition digital representation, said physical flavoring ingredient digital representation identifier being selected as a function of a value representative of the perceivable sweetness, bitterness, umami, licorice, lingering, sourness or saltiness associated to at least one physical flavoring ingredient digital representation identifier and the perceivable sweetness, bitterness, umami, licorice, lingering, sourness or saltiness set and
a step 315 of providing, upon a computer interface, the determined physical flavoring ingredient digital representation identifiers.

The step 305 of setting is performed, for example, by means 605 of setting such as shown in FIG. 6. Such means 605 of setting are, for example, a computer software executed upon a computing device, said software presenting controller characteristics for a computer interface, such as a keyboard 601 for example. Any type of computer interface may be used for this step 305 of setting, including but not limited to API inputs.

The value, set in this step 305 of setting, may be measured in Total Sweetness Brix Equivalent that refers to equivalent sucrose content in 100 g of product providing same sweetness perception than the one expected in the final product. This information can be translated by the algorithm in corresponding sweetness intensity or perceivable sweetness to be achieved

During this step 305 of setting, a value representative of a maximum sucrose content may be set as corresponding to the quantity of sucrose which can be used in the blend of sweeteners. This value may be used during the step 310 of determining.

During this step 305 of setting, a value representative of a maximum steviol equivalence may be set. This value may be used during the step 310 of determining as a limiting factor for the validity of the physical composition digital representation.

In particular embodiments, the step 305 of setting is performed via a graphic user interface (“GUI”), displaying on a screen 602 an input field configured to receive a numerical input from a user, said numerical input being representative of:
a desired perceivable sweetness, bitterness, umami, licorice, lingering, sourness or saltiness,
a minimum perceivable sweetness, bitterness, umami, licorice, lingering, sourness or saltiness or
a maximum perceivable sweetness, bitterness, umami, licorice, lingering, sourness or saltiness of a physical composition digital representation to be produced.

The latter two values may be embedded in the optimization algorithm by automatically defining acceptable ranges around an input desired value.

The step 310 of determining is performed, for example, by means 610 of determining such as shown in FIG. 6. Such means 610 of determining are, for example, a computer software executed upon a computing device. This step 310 of determining may be performed in a variety of ways.

In particular embodiments, physical flavoring ingredient digital representation identifiers are selected at random, and the perceivable sweetness, bitterness, umami, licorice, lingering, sourness or saltiness associated with the selected physical flavoring ingredient digital representation identifiers is computed. This perceivable sweetness, bitterness, umami, licorice, lingering, sourness or saltiness may then be compared with the set desired, minimum or maximum perceivable sweetness, bitterness, umami, licorice, lingering, sourness or saltiness. Depending on the output of this comparison, the physical composition digital representation digital representation identifier may be maintained and presented to a user for validation.

In particular embodiments, all combinations are tested (brute-force algorithm) and compared to the objective (set of desired, minimum, maximum sweetness+other constraints).

In particular embodiments, a random starting combination is generated, and the optimization algorithm is using gradient descend methodology to compare values of cost/desirability function at each iteration and determines the combination minimizing the cost function (or maximizing desirability function).

When several physical composition digital representation digital representation identifiers are valid, the physical composition digital representation digital representation identifiers may be ranked as a function of the associated perceivable sweetness, bitterness, umami, licorice, lingering, sourness or saltiness or a secondary ranking criterion.

The step 315 of providing is performed, for example, by means 620 of providing such as shown in FIG. 6. Such means 620 of providing is, for example, a computer software executed upon a computing device, said software presenting controller characteristics for a computer interface, such as a computer screen 602 for example.

In particular embodiments, such as shown in FIG. 4, the method 400 object of the present invention comprises:
a step 405 of associating, by a computing system, at least one secondary attribute with at least one physical flavoring ingredient digital representation identifier and
a step 410 of defining, for a physical composition digital representation to be produced, at least one threshold value for at least one secondary attribute,
   the step 310 of determining being performed as a function of the secondary attribute associated with at least one physical flavoring ingredient digital representation identifier.

The step 405 of associating is performed, for example, by a computer software associated to a computing device. During this step 405 of associating, a user or computer program may associate, in a database, digital representation identifiers of physical flavoring ingredients with attributes representative of a physical parameter of the physical flavoring ingredients. Such a secondary attribute may be, for example, an extraction method or a quality indicator.

The step 410 of defining is performed, for example, by a computer software associated to a computing device. This step 410 of defining may be performed similarly to the step 305 of setting. In variants, the step 410 of defining is performed by prompting a user, upon a computer interface, to select whether or not a particular secondary attribute must be present for physical flavoring ingredients, for example.

Depending on the threshold set, the step 310 of determining may evaluate the validity of generated physical composition digital representation digital representation identifiers.

In particular embodiments, such as shown in FIG. 4, the method 400 object of the present invention comprises a step 415 of assembling the physical composition represented by the physical composition digital representation. Such a step 415 of assembling may be performed according to any method known to a person skilled in the art of assembling physical flavoring chemical physical compositions.

In particular embodiments, such as shown in FIG. 4, the method 400 object of the present invention comprises, downstream of the step 305 of setting, a step 420 of calculating a maximum value for at least one attribute among:
a sodium chloride quantity,
a potassium chloride quantity,
a mono sodium glutamate quantity and/or
a ribotide quantity,
   at least one said calculated value being used during the step 310 of determining a physical composition digital representation.

The step 420 of calculating may be performed, for example, by a computer software run upon a computing system. During said step 420 of calculating, values corresponding to:
a sodium chloride quantity,
a potassium chloride quantity,
a mono sodium glutamate quantity and/or
a ribotide quantity,
   may be extracted from a correspondence database linking saltiness perception to ranges or values for the above attributes. The footprint of said ranges or values may then be used to match against the ranges or values for candidate ingredients and, should said candidate ingredients match said attribute ranges or values, those ingredients may be selected to form the physical composition digital representation. At least one such ingredient may be selected based upon secondary criteria for selection to be maximized or minimized, such as cost or renewable sourcing for said candidate ingredients.

FIG. 5 shows a particular embodiment of the system 500 object of the present invention. This physical composition digital representation sweetness, bitterness, umami, licorice, lingering, sourness or saltiness perception prediction system 500 comprises:
means 505 of inputting at least one physical flavoring ingredient digital representation identifier, said physical flavoring ingredient digital representation identifier being representative of a physical flavoring ingredient, said input defining a physical composition digital representation,
means 510 of defining for at least one physical flavoring ingredient digital representation identifier, a concentration of said physical flavoring ingredient in the physical composition digital representation,
means 515 of calculating, as a function of at least one concentration defined, a value representative of the perceivable sweetness, bitterness, umami, licorice, lingering, sourness or saltiness of the input physical composition digital representation, said calculated value being representative of the physico-chemical activation of taste receptors by the physical composition corresponding to the composition digital representation, and
means 520 of providing the calculated value representative of the perceivable sweetness, bitterness, umami, licorice, lingering, sourness or saltiness.

Examples of embodiments of the features of this system 500 are disclosed in regard of FIGS. 1 and 2.

FIG. 6 shows a particular embodiment of the system 600 object of the present invention. This physical composition digital representation sweetness, bitterness, umami, licorice, lingering, sourness or saltiness optimization system 600 comprises:
means 605 of setting a value representative of a desired perceivable sweetness, bitterness, umami, licorice, lingering, sourness or saltiness, minimum perceivable sweetness, bitterness, umami, licorice, lingering, sourness or saltiness or maximum perceivable sweetness, bitterness, umami, licorice, lingering, sourness or saltiness of a physical composition digital representation to be produced,
means 610 of determining at least one physical flavoring ingredient digital representation identifier, upon a computer interface, said physical flavoring ingredient digital representation identifier being representative of a physical flavoring ingredient, said input defining a physical composition digital representation, said physical flavoring ingredient digital representation identifier being selected as a function of a value representative of the perceivable sweetness, bitterness, umami, licorice, lingering, sourness or saltiness associated to at least one physical flavoring ingredient digital representation identifier and the perceivable sweetness, bitterness, umami, licorice, lingering, sourness or saltiness set and
means 615 of providing the determined physical flavoring ingredient digital representation identifiers.

Examples of embodiments of the features of this system 600 are disclosed in regard of FIGS. 3 and 4.

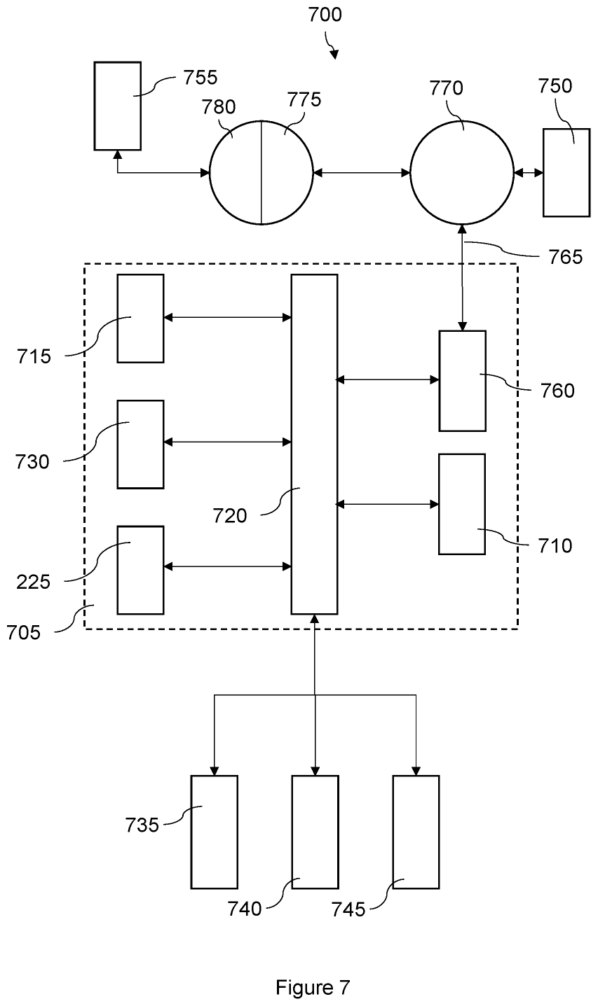

FIG. 7 represents a block diagram that illustrates an example computer system 700 with which may implement an embodiment of the present invention. Such a computer system 700 is also referred to as a computing system or a computing device in the present document. In the example of FIG. 7, a computer system 705 and instructions for implementing the disclosed technologies in hardware, software, or a combination of hardware and software, are represented schematically, for example as boxes and circles, at the same level of detail that is commonly used by persons of ordinary skill in the art to which this disclosure pertains for communicating about computer architecture and computer systems implementations.

The computer system 705 includes an input/output (IO) subsystem 720 which may include a bus and/or other communication mechanism(s) for communicating information and/or instructions between the components of the computer system 705 over electronic signal paths. The I/O subsystem 720 may include an I/O controller, a memory controller and at least one I/O port. The electronic signal paths are represented schematically in the drawings, for example as lines, unidirectional arrows, or bidirectional arrows.

At least one hardware processor 710 is coupled to the I/O subsystem 720 for processing information and instructions. Hardware processor 710 may include, for example, a general-purpose microprocessor or microcontroller and/or a special-purpose microprocessor such as an embedded system or a graphics processing unit (GPU) or a digital signal processor or ARM processor. Processor 710 may comprise an integrated arithmetic logic unit (ALU) or may be coupled to a separate ALU.

Computer system 705 includes one or more units of memory 725, such as a main memory, which is coupled to I/O subsystem 720 for electronically digitally storing data and instructions to be executed by processor 710. Memory 725 may include volatile memory such as various forms of random-access memory (RAM) or other dynamic storage device. Memory 725 also may be used for storing temporary variables or other intermediate information during execution of instructions to be executed by processor 710. Such instructions, when stored in non-transitory computer-readable storage media accessible to processor 710, can render computer system 705 into a special-purpose machine that is customized to perform the operations specified in the instructions.

Computer system 705 further includes non-volatile memory such as read only memory (ROM) 730 or other static storage device coupled to the I/O subsystem 720 for storing information and instructions for processor 710. The ROM 730 may include various forms of programmable ROM (PROM) such as erasable PROM (EPROM) or electrically erasable PROM (EEPROM). A unit of persistent storage 715 may include various forms of non-volatile RAM (NVRAM), such as FLASH memory, or solid-state storage, magnetic disk, or optical disk such as CD-ROM or DVD-ROM and may be coupled to I/O subsystem 720 for storing information and instructions. Storage 715 is an example of a non-transitory computer-readable medium that may be used to store instructions and data which when executed by the processor 710 cause performing computer-implemented methods to execute the techniques herein.

The instructions in memory 725, ROM 730 or storage 715 may comprise one or more sets of instructions that are organized as modules, methods, objects, functions, routines, or calls. The instructions may be organized as one or more computer programs, operating system services, or application programs including mobile apps. The instructions may comprise an operating system and/or system software; one or more libraries to support multimedia, programming or other functions; data protocol instructions or stacks to implement TCP/IP, HTTP or other communication protocols; file format processing instructions to parse or render files coded using HTML, XML, JPEG, MPEG or PNG; user interface instructions to render or interpret commands for a graphical user interface (GUI), command-line interface or text user interface; application software such as an office suite, internet access applications, design and manufacturing applications, graphics applications, audio applications, software engineering applications, educational applications, games or miscellaneous applications. The instructions may implement a web server, web application server or web client. The instructions may be organized as a presentation layer, application layer and data storage layer such as a relational database system using structured query language (SQL) or no SQL, an object store, a graph database, a flat file system or other data storage.

Computer system 705 may be coupled via I/O subsystem 720 to at least one output device 735. In one embodiment, output device 735 is a digital computer display. Examples of a display that may be used in various embodiments include a touch screen display or a light-emitting diode (LED) display or a liquid crystal display (LCD) or an e-paper display. Computer system 705 may include other type(s) of output devices 735, alternatively or in addition to a display device. Examples of other output devices 735 include printers, ticket printers, plotters, projectors, sound cards or video cards, speakers, buzzers or piezoelectric devices or other audible devices, lamps or LED or LCD indicators, haptic devices, actuators, or servos.

At least one input device 740 is coupled to I/O subsystem 720 for communicating signals, data, command selections or gestures to processor 710. Examples of input devices 740 include touch screens, microphones, still and video digital cameras, alphanumeric and other keys, keypads, keyboards, graphics tablets, image scanners, joysticks, clocks, switches, buttons, dials, slides.

Another type of input device is a control device 745, which may perform cursor control or other automated control functions such as navigation in a graphical interface on a display screen, alternatively or in addition to input functions. Control device 745 may be a touchpad, a mouse, a trackball, or cursor direction keys for communicating direction information and command selections to processor 710 and for controlling cursor movement on display 735. The input device may have at least two degrees of freedom in two axes, a first axis (e.g., x) and a second axis (e.g., y), that allows the device to specify positions in a plane. Another type of input device is a wired, wireless, or optical control device such as a joystick, wand, console, steering wheel, pedal, gearshift mechanism or other type of control device. An input device 740 may include a combination of multiple different input devices, such as a video camera and a depth sensor.

In another embodiment, computer system 705 may comprise an internet of things (IoT) device in which one or more of the output device 735, input device 740, and control device 745 are omitted. Or, in such an embodiment, the input device 740 may comprise one or more cameras, motion detectors, thermometers, microphones, seismic detectors, other sensors or detectors, measurement devices or encoders and the output device 735 may comprise a special-purpose display such as a single-line LED or LCD display, one or more indicators, a display panel, a meter, a valve, a solenoid, an actuator or a servo.

Computer system 705 may implement the techniques described herein using customized hard-wired logic, at least one ASIC or FPGA, firmware and/or program instructions or logic which when loaded and used or executed in combination with the computer system causes or programs the computer system to operate as a special-purpose machine. According to one embodiment, the techniques herein are performed by computer system 705 in response to processor 710 executing at least one sequence of at least one instruction contained in main memory 725. Such instructions may be read into main memory 725 from another storage medium, such as storage 715. Execution of the sequences of instructions contained in main memory 725 causes processor 710 to perform the process steps described herein. In alternative embodiments, hard-wired circuitry may be used in place of or in combination with software instructions.

The term “storage media” as used herein refers to any non-transitory media that store data and/or instructions that cause a machine to operation in a specific fashion. Such storage media may comprise non-volatile media and/or volatile media. Non-volatile media includes, for example, optical or magnetic disks, such as storage 715. Volatile media includes dynamic memory, such as memory 725. Common forms of storage media include, for example, a hard disk, solid state drive, flash drive, magnetic data storage medium, any optical or physical data storage medium, memory chip, or the like.

Storage media is distinct from but may be used in conjunction with transmission media. Transmission media participates in transferring information between storage media. For example, transmission media includes coaxial cables, copper wire and fiber optics, including the wires that comprise a bus of I/O subsystem 720. Transmission media can also take the form of acoustic or light waves, such as those generated during radio-wave and infra-red data communications.

Various forms of media may be involved in carrying at least one sequence of at least one instruction to processor 710 for execution. For example, the instructions may initially be carried on a magnetic disk or solid-state drive of a remote computer. The remote computer can load the instructions into its dynamic memory and send the instructions over a communication link such as a fiber optic or coaxial cable or telephone line using a modem. A modem or router local to computer system 705 can receive the data on the communication link and convert the data to a format that can be read by computer system 705. For instance, a receiver such as a radio frequency antenna or an infrared detector can receive the data carried in a wireless or optical signal and appropriate circuitry can provide the data to I/O subsystem 720 such as place the data on a bus. I/O subsystem 720 carries the data to memory 725, from which processor 710 retrieves and executes the instructions. The instructions received by memory 725 may optionally be stored on storage 715 either before or after execution by processor 710.

Computer system 705 also includes a communication interface 760 coupled to bus 720. Communication interface 760 provides a two-way data communication coupling to network link(s) 765 that are directly or indirectly connected to at least one communication networks, such as a network 770 or a public or private cloud on the Internet. For example, communication interface 760 may be an Ethernet networking interface, integrated-services digital network (ISDN) card, cable modem, satellite modem, or a modem to provide a data communication connection to a corresponding type of communications line, for example an Ethernet cable or a metal cable of any kind or a fiber-optic line or a telephone line. Network 770 broadly represents a local area network (LAN), wide-area network (WAN), campus network, internetwork, or any combination thereof. Communication interface 760 may comprise a LAN card to provide a data communication connection to a compatible LAN, or a cellular radiotelephone interface that is wired to send or receive cellular data according to cellular radiotelephone wireless networking standards, or a satellite radio interface that is wired to send or receive digital data according to satellite wireless networking standards. In any such implementation, communication interface 760 sends and receives electrical, electromagnetic, or optical signals over signal paths that carry digital data streams representing various types of information.

Network link 765 typically provides electrical, electromagnetic, or optical data communication directly or through at least one network to other data devices, using, for example, satellite, cellular, Wi-Fi, or BLUETOOTH technology. For example, network link 765 may provide a connection through a network 770 to a host computer 750.

Furthermore, network link 765 may provide a connection through network 770 or to other computing devices via internetworking devices and/or computers that are operated by an Internet Service Provider (ISP) 775. ISP 775 provides data communication services through a world-wide packet data communication network represented as internet 780. A server computer 755 may be coupled to internet 780. Server 755 broadly represents any computer, data center, virtual machine, or virtual computing instance with or without a hypervisor, or computer executing a containerized program system such as DOCKER or KUBERNETES. Server 755 may represent an electronic digital service that is implemented using more than one computer or instance and that is accessed and used by transmitting web services requests, uniform resource locator (URL) strings with parameters in HTTP payloads, API calls, app services calls, or other service calls. Computer system 705 and server 755 may form elements of a distributed computing system that includes other computers, a processing cluster, server farm or other organization of computers that cooperate to perform tasks or execute applications or services. Server 755 may comprise one or more sets of instructions that are organized as modules, methods, objects, functions, routines, or calls. The instructions may be organized as one or more computer programs, operating system services, or application programs including mobile apps. The instructions may comprise an operating system and/or system software; one or more libraries to support multimedia, programming or other functions; data protocol instructions or stacks to implement TCP/IP, HTTP or other communication protocols; file format processing instructions to parse or render files coded using HTML, XML, JPEG, MPEG or PNG; user interface instructions to render or interpret commands for a graphical user interface (GUI), command-line interface or text user interface; application software such as an office suite, internet access applications, design and manufacturing applications, graphics applications, audio applications, software engineering applications, educational applications, games or miscellaneous applications. Server 755 may comprise a web application server that hosts a presentation layer, application layer and data storage layer such as a relational database system using structured query language (SQL) or no SQL, an object store, a graph database, a flat file system or other data storage.

Computer system 705 can send messages and receive data and instructions, including program code, through the network(s), network link 765 and communication interface 760. In the Internet example, a server 755 might transmit a requested code for an application program through Internet 780, ISP 775, local network 770 and communication interface 760. The received code may be executed by processor 710 as it is received, and/or stored in storage 715, or other non-volatile storage for later execution.

The execution of instructions as described in this section may implement a process in the form of an instance of a computer program that is being executed and consisting of program code and its current activity. Depending on the operating system (OS), a process may be made up of multiple threads of execution that execute instructions concurrently. In this context, a computer program is a passive collection of instructions, while a process may be the actual execution of those instructions. Several processes may be associated with the same program; for example, opening up several instances of the same program often means more than one process is being executed. Multitasking may be implemented to allow multiple processes to share processor 710. While each processor 710 or core of the processor executes a single task at a time, computer system 705 may be programmed to implement multitasking to allow each processor to switch between tasks that are being executed without having to wait for each task to finish. In an embodiment, switches may be performed when tasks perform input/output operations, when a task indicates that it can be switched, or on hardware interrupts. Time-sharing may be implemented to allow fast response for interactive user applications by rapidly performing context switches to provide the appearance of concurrent execution of multiple processes simultaneously. In an embodiment, for security and reliability, an operating system may prevent direct communication between independent processes, providing strictly mediated and controlled inter-process communication functionality.

In the description below, the terms “physical network device” designate any element of hardware connected upon a computer network from which said element draws connectivity to other physical and/or virtual network devices.

In the description below, the terms “computer network” designate any embodiment of the network 770 disclosed in regard of FIG. 7.

In the description below, the terms “connectivity usage” may refer to any metric typically used to monitor the usage of a communication link, such as a value representative of the connectivity usage corresponds to a response time of a distant device, time to each a DNS server, loss packets, time to establish a SSL connection, a bandwidth use or a latency for example.

As it is understood, the present invention may be integrated into an optimization algorithm with two modes: one dedicated to Application Technologists, the other designed for Flavorists.

The first mode allows to identify best physical composition digital representation and related dosage among existing physical flavoring ingredients regarding physical composition digital representation requirements. This identification can be done thanks to a brute-forced methodology. At first some physical composition digital representation validity threshold can be used to reduce the number of physical composition digital representations to screen to the relevant ones. For example, physical composition digital representations containing ingredients which are banned in the targeted countries are automatically dropped from the search. Then all possible dosages are simulated and after several steps of filtering, the most relevant physical composition digital representations with a dosage recommendation are shown to the user. Filtering can be based on several aspects: physical composition digital representations which overcome maximum usage limits for some ingredients in the targeted selling countries are dropped. Cost should not be higher than a target. Finally cost/quality ratio is also considered.

The second mode, which is activated only for flavorists, allows to generate new combinations of sweeteners. To do so, a so called “desirability function” can been designed. This function describes how good a combination of sweeteners is, knowing validity requirements and the concentration of individual sweeteners. This function includes in addition some business knowledge like key performance indicators in the context of sugar reduced sweet applications, such as the ratio between sweetness and licorice perception. The higher the value of desirability, the better the combination of sweeteners is. This function being not convex and presenting multiple optimal points a multi-start logic may be applied to make sure the algorithm identifies all relevant sweetener blends.

It should be understood that the present invention allows for taste perception prediction and, based upon this prediction capacity, allows for automatic physical composition digital representation design or physical composition digital representation optimization. In the case of optimization, the optimization may result from the input of an initial physical composition digital representation of ingredients or from the input of a target for taste perception resulting in the autonomous physical composition digital representation of ingredients to match said target. Furthermore, secondary targets may be set, such as cost targets, secondary taste targets (umami, bitterness, lingering or licorice) to be minimized or maximized or targets representative of regional taste variations, said targets being used during the optimization of the physical composition digital representation.

The use of the optimization capacities of the present invention may differ as such:
in a perceivable sugar reduction objective, a user may set a physical composition digital representation of ingredients, the method providing alternative ingredients reducing the sugar physical composition digital representation of the set physical composition digital representation without changing the sweetness perception achieved, in which case a user may select said alternative ingredient to optimize the physical composition digital representation,
in a perceivable salt reduction objective, a user may set a target saltiness for a physical composition digital representation of ingredients to be selected, the perceivable saltiness set being converted into sub-attributes such as:
   a sodium chloride quantity,
   a potassium chloride quantity,
   a mono sodium glutamate quantity and/or
   a ribotide quantity,
      these sub-attributes being used to select, among a database of selectable ingredients, at least one ingredient such that the sum of said selectable ingredients matches the set target in terms of saltiness perception with less salt in the physical composition digital representation.

1. Physical composition digital representation sweetness, bitterness, umami, licorice, lingering, sourness or saltiness perception prediction method, comprising:
a step of inputting at least one physical flavoring ingredient digital representation identifier, upon a computer interface, said physical flavoring ingredient digital representation identifier being representative of a physical flavoring ingredient, said input defining a physical composition digital representation,
a step of defining, upon a computer interface, for at least one physical flavoring ingredient digital representation identifier, a concentration of said physical flavoring ingredient in the physical composition digital representation,
a step of calculating, by a computing system, as a function of at least one concentration defined, a value representative of the perceivable sweetness, bitterness, umami, licorice, lingering, sourness or saltiness of the input physical composition digital representation, said calculated value being representative of the physico-chemical activation of taste receptors by the physical composition corresponding to the composition digital representation, and
a step of providing, upon a computer interface, the calculated value representative of the perceivable sweetness, bitterness, umami, licorice, lingering, sourness or saltiness.

2. Method according to claim 1, in which the step of calculating is configured to operate a nth order polynomial function as a function of at least one defined concentration of physical flavoring ingredient in the physical composition digital representation.

3. Method according to claim 2, which comprises:
a step of initialization, upon a computer interface, of physical flavoring ingredient digital representation identifiers, said input defining a physical composition digital representation,
a step of generating, by a computing system, a list of physical composition digital representation digital representation identifiers, each physical composition digital representation digital representation identifiers being representative of a physical composition digital representation,
a step of producing at least one generated physical composition digital representation,
a step of acquisition, upon a computer interface, of values representative of the perceived sweetness, bitterness, umami, licorice, lingering, sourness or saltiness of at least one generated physical composition digital representation and
a step of computing, by a computing system, coefficients in a nth order polynomial function using the input physical flavoring ingredient digital representation identifiers as variables,
said coefficients being used during the step of calculating.

4. Method according to claim 3, which further comprises a step of setting, upon a computer interface, for at least one physical flavoring ingredient digital representation identifier, a value representative of the concentration of said physical flavoring ingredient in the input physical composition digital representation, the steps of producing and computing being performed as a function of said concentration.

5. Method according to claim 3, which comprises, downstream of the step of generating, a step of physical composition digital representation digital representation identifier generation optimization, the step of producing being performed as a function of the output of said step of optimization.

6. Method according to claim 3, which comprises a step of constructing a database of physical flavoring ingredient interaction impact value upon sweetness, bitterness, umami, licorice, lingering, sourness or saltiness perception, said impact value:
being obtained as a function of the coefficients obtained during the step of computing and of the physical flavoring ingredient digital representation identifiers associated with these coefficients, and
used during the step of calculating.

7. Method according to claim 1, which comprises a step of determining, by a computing system, at least one replacement physical flavoring ingredient digital representation identifier for at least one input physical flavoring ingredient digital representation identifier, the step of providing being configured to provide the determined physical flavoring ingredient digital representation identifier.

8. Method according to claim 1, which comprises a step of selecting a physical composition application identifier for the physical composition, the step of calculating being configured to determine a prediction as a function of the physical composition application identifier selected.

9. Method according to claim 1, which comprises a step of physical ingredients interaction parameter values database construction, in which at least two physical ingredients digital identifiers are associated to at least one physical interaction parameter value representative of a quantified synergistic, neutral or antagonistic interaction between the corresponding physical ingredients.

10. Method according to claim 1, which comprises, downstream of the step of defining, a step of attributing, for at least one input physical flavoring ingredient digital representation identifier, a value for at least one attribute among:
a sodium chloride quantity,
a potassium chloride quantity,
a mono sodium glutamate quantity and/or
a ribotide quantity,
at least one said value being used during the step of calculating to calculate a value representative of the perceivable saltiness, umami or bitterness of the input physical composition digital representation.

11. Physical composition digital representation sweetness, bitterness, umami, licorice, lingering, sourness or saltiness optimization method, comprising:
a step of setting, upon a computer interface, a value representative of a desired perceivable sweetness, bitterness, umami, licorice, lingering, sourness or saltiness, minimum perceivable sweetness, bitterness, umami, licorice, lingering, sourness or saltiness or maximum perceivable sweetness, bitterness, umami, licorice, lingering, sourness or saltiness of a physical composition digital representation to be produced,
a step of determining, by a computing system, at least one physical flavoring ingredient digital representation identifier, upon a computer interface, said physical flavoring ingredient digital representation identifier being representative of a physical flavoring ingredient, said input defining a physical composition digital representation, said physical flavoring ingredient digital representation identifier being selected as a function of a value representative of the perceivable sweetness, bitterness, umami, licorice, lingering, sourness or saltiness associated to at least one physical flavoring ingredient digital representation identifier and the perceivable sweetness, bitterness, umami, licorice, lingering, sourness or saltiness set and
a step of providing, upon a computer interface, the determined physical flavoring ingredient digital representation identifiers.

12. Method according to claim 11, which comprises:
a step of associating, by a computing system, at least one secondary attribute with at least one physical flavoring ingredient digital representation identifier and
a step of defining, for a physical composition digital representation to be produced, at least one threshold value for at least one secondary attribute,
the step of determining being performed as a function of the secondary attribute associated with at least one physical flavoring ingredient digital representation identifier.

13. Method according to claim 11, in which at least one value set, during the step of setting corresponds to:
a total sweetness brix equivalent, referring to equivalent sucrose content in 100 g of product providing same sweetness perception than the one the physical composition digital representation to be determined,
a value representative of a maximum sucrose content
a maximum steviol equivalence.

14. Method according to claim 11, which comprises, downstream of the step of setting, a step of calculating a maximum value for at least one attribute among:
a sodium chloride quantity,
a potassium chloride quantity,
a mono sodium glutamate quantity and/or
a ribotide quantity,
at least one said calculated value being used during the step of determining a physical composition digital representation.

15. Method according to claim 1, which comprises a step of assembling the physical composition represented by the physical composition digital representation.

16. Physical composition digital representation sweetness, bitterness, umami, licorice, lingering, sourness or saltiness perception prediction system, comprising:
means of inputting at least one physical flavoring ingredient digital representation identifier, said physical flavoring ingredient digital representation identifier being representative of a physical flavoring ingredient, said input defining a physical composition digital representation,
means of defining for at least one physical flavoring ingredient digital representation identifier, a concentration of said physical flavoring ingredient in the physical composition digital representation,
means of calculating, as a function of at least one concentration defined, a value representative of the perceivable sweetness, bitterness, umami, licorice, lingering, sourness or saltiness of the input physical composition digital representation, said calculated value being representative of the physico-chemical activation of taste receptors by the physical composition corresponding to the composition digital representation and
means of providing the calculated value representative of the perceivable sweetness, bitterness, umami, licorice, lingering, sourness or saltiness.

17. Physical composition digital representation sweetness, bitterness, umami, licorice, lingering, sourness or saltiness optimization system, comprising:
means of setting a value representative of a desired perceivable sweetness, bitterness, umami, licorice, lingering, sourness or saltiness, minimum perceivable sweetness, bitterness, umami, licorice, lingering, sourness or saltiness or maximum perceivable sweetness, bitterness, umami, licorice, lingering, sourness or saltiness of a physical composition digital representation to be produced,
means of determining at least one physical flavoring ingredient digital representation identifier, upon a computer interface, said physical flavoring ingredient digital representation identifier being representative of a physical flavoring ingredient, said input defining a physical composition digital representation, said physical flavoring ingredient digital representation identifier being selected as a function of a value representative of the perceivable sweetness, bitterness, umami, licorice, lingering, sourness or saltiness associated to at least one physical flavoring ingredient digital representation identifier and the perceivable sweetness, bitterness, umami, licorice, lingering, sourness or saltiness set and
means of providing the determined physical flavoring ingredient digital representation identifiers.
